(19) United States
(12) Patent Application Publication
(10) Pub. No.: US 2024/0091532 A1
(43) Pub. Date: Mar. 21, 2024

(54) DEVICE FOR CARRYING OUT A TVNS TREATMENT

(71) Applicant: tVNS Technologies GmbH, Erlangen (DE)

(72) Inventor: Sven-Erik BOLZ, Buckenhof (DE)

(21) Appl. No.: 18/274,613

(22) PCT Filed: Feb. 1, 2022
(86) PCT No.: PCT/EP2022/052312

§ 371 Date: Jul. 27, 2023

(30) Foreign Application Priority Data
Feb. 2, 2021 (DE) 10 2021 102 401.3

(51) Int. Cl. A61N 1/36; A61N 1/04

(52) U.S. Cl. CPC A61N 1/36025; A61N 1/0456; A61N 1/36031; A61N 1/36034

(57) ABSTRACT

The present invention relates to a device for carrying out a tVNS treatment with at least one electrode for generating a stimulation pulse. The device has at least one detection means which is designed to detect a patient movement, and the device has an open-loop or closed-loop control unit, designed to initiate the output of stimulation pulses through the electrode if a patient movement detected by the detection device reaches or exceeds a predetermined threshold value.

The present invention relates to a device for carrying out a tVNS treatment.

What is known as transcutaneous vagus nerve stimulation (also referred to in the following as tVNS) is known from the prior art as a treatment method which is based on a branch of the vagus nerve, specifically the ramus auricularis nervi vagi (RANV), being stimulated transcutaneously with electrical pulses. The method is used for example in the treatment of drug-resistant epilepsy (DRE) and refractory depression.

The treatment takes place using a device which generates electrical pulses which are released through the skin, to the mentioned branch of the vagus nerve, by an ear electrode which is worn like headphones.

In the case of rehabilitation of stroke patients, conventionally an invasive vagus nerve stimulation is used, in which the stimulators are implanted directly on the vagus nerve. However, on account of the invasive nature of this treatment, this procedure is unsuitable for some patient groups.

The object of the present invention is therefore that of reducing or even entirely overcoming the disadvantages of the prior art. In particular, the object of the present invention is that of providing a possibility for rehabilitation of people with reduced mobility, in particular stroke patients, which can be tolerated by as far as possible all patients.

This object is achieved by a device having the features of claim 1.

According thereto, a device for carrying out a tVNS treatment, comprising at least one electrode for generating a stimulation pulse, is provided, wherein the device has at least one detection means which is configured to detect a patient movement, and the device has an open-loop or closed-loop control unit, configured to initiate the output of stimulation pulses by the electrode if a patient movement detected by the detection means reaches or exceeds a predetermined threshold value.

In other words, the vagus nerve stimulation is intended to occur and/or take place as far as possible simultaneously with the patient movement or the attempted patient movement, in order to thereby make optimal use of the neuronal plasticity during the rehabilitation. As soon as the patient for example initiates or attempts a movement of their arm, the movement can be detected by the detection means, which is worn by the patient for example as a wristband or other wearable device, and the vagus nerve stimulation can be connected, in a supporting manner.

Preferably, the open-loop or closed-loop control unit is configured to automatically initiate the output of stimulation pulses by the electrode when the patient movement detected by the detection means reaches or exceeds a predetermined threshold value. Alternatively or additionally, a user can also manually initiate the output of stimulation pulses by the electrode when there has been a corresponding indication that the patient movement detected by the detection means reaches or exceeds a predetermined threshold value. It would also be conceivable for a user to be able to set or adjust the output of stimulation pulses during an ongoing treatment.

Preferably, the detection means is configured to measure the patient movement in real time, and the open-loop or closed-loop control unit is configured to control the output of stimulation pulses by the electrode in such a way that this occurs and/or takes place simultaneously with the detected patient movement.

In this case, the open-loop or closed-loop control unit can be configured to control the output of stimulation pulses by the electrode in such a way that this takes place continuously or intermittently during a portion of the duration or the entire duration of the detected patient movement. For example, the vagus nerve stimulation can in particular take place during the start of the movement or during the entire duration of the detected movement.

It has been found to be advantageous in practice if the open-loop or closed-loop control unit is configured to cause the output of stimulation pulses by the electrode having a pulse width of approx. 0.1 milliseconds, a frequency of 25 Hz, and an intensity of between 1 and 3.2 mA, preferably approx. 1.4 mA. The intensity of the stimulation is set for example to a maximum value that can be tolerated by the patient. However, the parameters of the vagus nerve stimulation can be flexibly adjusted according to the application or patient group, and the values mentioned above merely represent an example.

According to an advantageous embodiment of the invention, the open-loop or closed-loop control unit is configured to adjust or set the output of stimulation pulses by the electrode during a portion of the duration or the entire duration of the detected patient movement, on the basis of further physiological parameters of the patient and/or external parameters. For example, during the stimulation it is possible to react in a flexible manner to changes in the breathing rate, blood pressure, muscle tone or other patient parameters and/or also external parameters such as the ambient temperature, the weather, etc.

Furthermore, it has been found to be helpful in practice if the open-loop or closed-loop control unit is configured to end the output of stimulation pulses by the electrode when the patient movement detected by the detection means exceeds a predetermined threshold value and/or a predetermined time period has elapsed. In this way, the vagus nerve stimulation is preferably temporally linked to or coincides with the neuronal activity of the patient when performing the movement.

According to an embodiment of a device according to the invention, the detection means is configured to be worn by a patient, preferably by means of a wristband, and to transmit measurement data directly to a stimulation unit of the device that comprises the electrode, or an interposed processing unit, for example a mobile terminal.

In other words, a device according to the invention for example comprises a stimulation unit that comprises the electrode and the open-loop or closed-loop control unit, and a detection means formed separately therefrom. In this case, the detection means transmits data directly to the stimulation unit.

According to another embodiment, a device according to the invention for example comprises a stimulation unit that comprises the electrode, and a detection means formed separately therefrom. In addition, an open-loop or closed-loop control unit is provided in a processing unit, for example on a mobile terminal such as a smartphone or tablet. In this case, the detection means and the stimulation unit transmit data to the processing unit. The transmitted data are processed in the processing unit, and the stimulation unit is controlled on the basis of the data transmitted from the detection means.

Another aspect of the invention relates to a use of a device according to the invention in the rehabilitation of people with reduced mobility, in particular of stroke patients.

In this case, the device is preferably used over several weeks, preferably 6 weeks, several times per week, preferably three times per week. However, these specifications are given purely by way of example, and the application can be adjusted flexibly to different application cases.

Another aspect of the invention relates to a computer program product which prompts or enables a computer or a mobile terminal to carry out the following steps:
detecting a patient movement using a detection means;
open-loop or closed-loop control of a stimulation unit comprising an electrode for carrying out tVNS treatment in such a way that the output of stimulation pulses by the electrode is initiated if a patient movement detected by the detection means reaches or exceeds a predetermined threshold value.

In other words, a conventional device for carrying out a tVNS treatment can be retrofitted to form a device according to the present invention, by means of a computer program product according to the invention.

A program product according to the invention can also be used within the context of a processing unit formed by a mobile terminal.

Further features, advantages and effects of the present invention emerge from the following description.

For example, it can be provided, according to one embodiment, that the device for carrying out a tVNS treatment is configured comprising at least one electrode for generating a stimulation pulse, wherein the device has at least one detection means which is configured to detect one or more parameter values, wherein the device has an open-loop or closed-loop control unit which is suitable for setting one or more parameters of the stimulation pulse emitted by the electrode, depending on the detected parameter value(s).

After the initiation of a tVNS treatment, on account of the detection of a patient movement, the tVNS treatment can preferably be flexibly adjusted to the individual needs of the patients. Thus, in a manner deviating from the (non-adaptive) tVNS devices known from the prior art, not always the same stimulation is carried out. Rather, said stimulation is dependent, with respect to one or more parameters, such as duration or strength of the pulse, on one or more parameter values which are measured by a sensor means, i.e. by the detection means.

Thus, the essential advantage can be achieved that the stimulation, and thus also the treatment success, can take place in a manner individual to the patient, and/or that parameters that may influence the stimulation, such as room temperature, etc., can also be included in the stimulation, which increases the treatment success compared with an inflexible stimulation course, i.e. stimulation protocol.

The detection means are preferably configured in the form of one or more sensors.

The detection means can be configured to measure the parameter value(s), such as the patient's heart rate, in real time, and the open-loop or closed-loop control unit can be configured to set the stimulation pulse depending on the parameter value(s) measured in real time. In this case, storing of the measured parameter value is not essential. Rather, for example the (not stored) heart rate serves as an input variable, on the basis of which the stimulation pulse is then set.

According to a further embodiment, the detection means is connected to a memory, such that the parameter values measured by the detection means are stored in the memory, and the open-loop or closed-loop control unit is configured to set the stimulation pulse and/or a threshold value depending on the parameter value(s) stored in the memory. For example, the parameter can specify the magnitude of patient movements, or their movement level.

It would be conceivable, for example, for the heart rate to be ascertained and stored over a longer period of time, and then the stimulation pulse to be output on the basis of the stored values.

These may be vital parameters or patient parameters, or external parameters which are not related to the patient.

By way of example, the patient parameter(s) may be the patient's heart rate, at least one parameter that can be determined by means of an EEG sensor, a parameter relating to breathing, or a parameter relating to the sympathovagal balance, or a parameter relating to the patient movement, or a combination of the above-mentioned parameters.

As stated, alternatively or additionally it may be the case that no patient-related parameters are included in the open-loop or closed-loop control of the stimulation pulse, such as the room temperature, the air pressure, or other external, i.e. not patient-specific, physical variables.

Closed-loop control is also conceivable and covered by the invention.

It is thus conceivable, for example, for the closed-loop control unit to be configured to actuate the electrode in such a way that the parameter value detected by the detection means is regulated to a target value or into a target value range by means of the stimulation pulse. By way of example, an embodiment could be configured in such a way that tVNS influences the autonomous tone, i.e. the sympathovagal balance. If this tone is detected e.g. by means of pupillometry or skin conductance sensors, the closed-loop control unit can be configured in such a way that it specifically influences the stimulation and thus adjusts the tone to the current requirement in the form of a target value or target value range.

In a further embodiment of the invention, the open-loop or closed-loop control unit is configured to match the stimulation pulse(s) to periodically occurring physiological, in particular neurological, processes of the patient.

It is thus conceivable, for example, for the detection means to be suitable for detecting the patient's breathing, and for the open-loop or closed-loop control unit to be configured to match or trigger the stimulation pulses to the breathing signal.

According to a method for carrying out a tVNS treatment it can be provided that a stimulation pulse is generated by means of at least one electrode, preferably by means of an ear electrode, wherein a patient movement is detected by the detection means and the output of the stimulation pulses by the electrode is initiated if a patient movement detected by the detection means reaches or exceeds a predetermined threshold value.

Preferred features of the method are found in the features according to the method, of dependent claims 2 to 9. Preferred features of a computer program product according to the invention, in particular an app, are also found in the features according to the method, of dependent claims 2 to 9.

At this point it is noted that the terms “a/an” and “one” to not necessarily refer to exactly one of the elements, even if this constitutes a possible embodiment, but rather can also denote a plurality of the elements. Likewise, the use of the plural also includes the presence of the element in question in the singular, and, vice versa, the singular also includes a plurality of the elements in question.

Further details and advantages of the invention are explained in greater detail on the basis of an embodiment that is described in the following.

The embodiment relates to a device for carrying out a tVNS treatment which comprises one or more electrodes, preferably ear electrodes, for outputting stimulation pulses.

The vagus nerve stimulation is based on a branch of the vagus nerve, in the form of the ramus auricularis nervi vagi (RANV), treating the skin of the outer ear, in the region of the concha, in a sensory manner.

The RANV can be stimulated transcutaneously, i.e. through the skin, by means of the electrode, using electrical pulses. The stimulation of the RANV causes excitation of the vagus nerve which, as in the case of conventional (not transcutaneous) VNS, reaches higher centers of the brain via the brain stem.

The device generates electrical pulses and can be the size of a smartphone.

One or two ear electrodes can be used. The electrodes output the pulses through the skin, to the branch of the vagus nerve. The ear electrodes can be configured in such a way that these comprise loudspeakers, such that the patient can listen to music or the like during the treatment.

In contrast to a known device for carrying out a tVNS, the device according to the invention is a device, the stimulation of which is preferably initiated or triggered automatically by means of detection that a patient movement has exceeded a threshold value. The stimulation by the device preferably takes place simultaneously with the movement being carried out by the patient.

Instead of the stimulation being triggered automatically upon detection that a patient movement has exceeded a threshold value, it can also be provided, according to the invention, that a user is made aware, by means of an acoustic and/or optical output, that it has been detected that the patient movement has exceeded the threshold value, and the user is prompted to initiate and/or to end the stimulation manually.

A fundamental concept of the invention therefore consists in temporally synchronizing the transcutaneous stimulation of the vagus nerve with the (attempted) performance of a movement by the patient. Since the stimulation of the vagus nerve takes place in a non-invasive manner, a device according to the invention is suitable for almost all patient groups.

In contrast to a known device for carrying out a tVNS, the device according to the invention is furthermore preferably a device for adaptive tVNS, i.e. a device, the stimulation protocol of which is not always identical, but rather is dependent on one or more patient-specific and/or non-patient-specific parameters.

A further concept of the present invention therefore consists in extending a conventional tVNS stimulation device by means of various sensors, which serve to control or change the stimulation parameters.

The response to an event is cited as a first embodiment:

For example, it is known that the patient's heart rate increases shortly before an epileptic seizure. By means of an ECG sensor, the stimulation device according to the invention is capable of monitoring the profile of the heart rate and adjusting its stimulation to the current heart rate.

The synchronization of the output pulses with periodically occurring processes of the patient is cited as a second embodiment:

Certain neurological processes are subject to a certain amount of periodicity. Thus, for example, the tVNS device according to the invention can be synchronized to the physiological rhythm by means of an EEG sensor, and thus the therapeutic effect can be dramatically improved, because it always provides treatment in the patient's sensitive phase.

In a further example, the detection means is a breathing sensor which is configured to measure the periodic state of the sympathovagal system. The breathing sensor provides the periodic state of the sympathovagal system, and the tVNS device according to the invention is triggered upon this breathing signal.

In a further example, a closed-loop design, i.e. closed-loop control of a parameter to a target value or in a target value range, is conceivable:

By way of example, the tVNS influencing the autonomous tone, more precisely the sympathovagal balance, can be cited as an example of this. If this tone is detected for example by means of pupillometry or skin conductance sensors, the stimulation can be specifically influenced and thus adjusted to the current requirement of the patient. In this way, the tone can be kept in a particular target value range, optionally individual to the patient, wherein the stimulation pulse(s) serve(s) as the “actuator”.

It is noted that the above-mentioned embodiments to not restrict the invention. In principle, a plurality of sensors is conceivable: All vital parameters are in principle suitable, but also other parameters, such as movement, temperature, or pressure.

1. Device for carrying out a tVNS treatment, comprising at least one electrode for generating a stimulation pulse, wherein the device comprises at least one detection means which are configured to detect a patient movement, and an open-loop or closed-loop control unit, configured to initiate the output of stimulation pulses by the electrode if a patient movement detected by the detection means reaches or exceeds a predetermined threshold value.

2. Device according to claim 1, wherein the detection means are configured to measure the patient movement in real time, and the open-loop or closed-loop control unit is configured to control the output of stimulation pulses by the electrode in such a way to take place simultaneously with the detected patient movement.

3. Device according to claim 1, wherein the open-loop or closed-loop control unit is configured to control the output of stimulation pulses by the electrode in such a way to take place continuously or intermittently during a portion of the duration or the entire duration of the detected patient movement.

4. Device according to claim 1, wherein the open-loop or closed-loop control unit is configured to cause the output of stimulation pulses by the electrode having a pulse width of approx. 0.1 milliseconds, a frequency of 25 Hz, and an intensity of between 1 and 3.2 mA, preferably approx. 1.4 mA.

5. Device according to claim 1, wherein the open-loop or closed-loop control unit is configured to adjust or set the output of stimulation pulses by the electrode during a portion of the duration or the entire duration of the detected patient movement, on the basis of further physiological parameters of the patient.

6. Device according to claim 1, wherein the open-loop or closed-loop control unit is configured to end the output of stimulation pulses by the electrode when the patient movement detected by the detection means exceeds a predetermined threshold value and/or a predetermined time period has elapsed.

7. Device according to claim 1, wherein the detection means are configured to be worn by a patient, preferably by a wristband, and to transmit measurement data directly to a stimulation unit of the device that comprises the electrode, or an interposed processing unit, for example a mobile terminal.

8. Method for the rehabilitation of people with reduced mobility, in particular of stroke patients, comprising monitoring a patient with the device according to claim 1.

9. Method according to claim 8, wherein the device is used over several weeks, preferably 6 weeks, several times per week, preferably three times per week.

10. Computer program product which prompts or enables a computer or a mobile terminal to carry out the following steps:
detecting a patient movement using detection means; and
open-loop or closed-loop control of a stimulation unit comprising an electrode for carrying out tVNS treatment in such a way the output of stimulation pulses by the electrode is initiated if a patient movement detected by the detection means reaches or exceeds a predetermined threshold value.

11. Device according to claim 2, wherein the open-loop or closed-loop control unit is configured to control the output of stimulation pulses by the electrode in such a way to take place continuously or intermittently during a portion of the duration or the entire duration of the detected patient movement.

12. Device according to claim 11, wherein the open-loop or closed-loop control unit is configured to cause the output of stimulation pulses by the electrode having a pulse width of approx. 0.1 milliseconds, a frequency of 25 Hz, and an intensity of between 1 and 3.2 mA, preferably approx. 1.4 mA.

13. Device according to claim 3, wherein the open-loop or closed-loop control unit is configured to cause the output of stimulation pulses by the electrode having a pulse width of approx. 0.1 milliseconds, a frequency of 25 Hz, and an intensity of between 1 and 3.2 mA, preferably approx. 1.4 mA.

14. Device according to claim 2, wherein the open-loop or closed-loop control unit is configured to cause the output of stimulation pulses by the electrode having a pulse width of approx. 0.1 milliseconds, a frequency of 25 Hz, and an intensity of between 1 and 3.2 mA, preferably approx. 1.4 mA.

15. Device according to claim 14, wherein the open-loop or closed-loop control unit is configured to adjust or set the output of stimulation pulses by the electrode during a portion of the duration or the entire duration of the detected patient movement, on the basis of further physiological parameters of the patient.

16. Device according to claim 13, wherein the open-loop or closed-loop control unit is configured to adjust or set the output of stimulation pulses by the electrode during a portion of the duration or the entire duration of the detected patient movement, on the basis of further physiological parameters of the patient.

17. Device according to claim 12, wherein the open-loop or closed-loop control unit is configured to adjust or set the output of stimulation pulses by the electrode during a portion of the duration or the entire duration of the detected patient movement, on the basis of further physiological parameters of the patient.

18. Device according to claim 11, wherein the open-loop or closed-loop control unit is configured to adjust or set the output of stimulation pulses by the electrode during a portion of the duration or the entire duration of the detected patient movement, on the basis of further physiological parameters of the patient.

19. Device according to claim 4, wherein the open-loop or closed-loop control unit is configured to adjust or set the output of stimulation pulses by the electrode during a portion of the duration or the entire duration of the detected patient movement, on the basis of further physiological parameters of the patient.

20. Device according to claim 3, wherein the open-loop or closed-loop control unit is configured to adjust or set the output of stimulation pulses by the electrode during a portion of the duration or the entire duration of the detected patient movement, on the basis of further physiological parameters of the patient.
